(19) United States
(12) Patent Application Publication
(10) Pub. No.: US 2019/0076440 A1
(43) Pub. Date: Mar. 14, 2019

(54) TREATMENT OF TISSUE DISORDERS

(71) Applicant: University of Newcastle Upon Tyne, Newcastle Upon Tyne (GB)

(72) Inventors: Raymond BOOT-HANDFORD, Newcastle Upon Tyne (GB); Michael BRIGGS, Newcastle Upon Tyne (GB); Eileithyia SWANTON, Newcastle Upon Tyne (GB); Ewa MULARCZYK, Newcastle Upon Tyne (GB); Lorna MULLAN, Newcastle Upon Tyne (GB)

(21) Appl. No.: 16/085,149

(22) PCT Filed: Mar. 15, 2017
(86) PCT No.: PCT/GB2017/050710

§ 371 Date: Sep. 14, 2018

(30) Foreign Application Priority Data
Mar. 15, 2016 (GB) 1604359.8

(51) Int. Cl. A61K 31/55; A61P 19/08; A61K 9/00; A61P 27/02

(52) U.S. Cl. CPC A61K 31/55; A61P 19/08; A61P 27/02; A61K 9/0029; A61K 9/0053

(57) ABSTRACT

Aspects of the present invention relate inter alia to the treatment of disorders which are characterised by inappropriate intracellular protein accumulation using carbamazepine and/or a carbamazepine-like compound. Also described herein are methods of treating such disorders comprising administering a composition comprising carbamazepine or a carbamazepine-like compound to a subject in need thereof. Exemplary disorders include for example connective tissue disorders.

### FIELD OF THE INVENTION

Aspects of the present invention relate inter alia to the treatment of disorders which are characterised by inappropriate intracellular protein accumulation using carbamazepine and/or a carbamazepine-like compound. Also described herein are methods of treating such disorders comprising administering a composition comprising carbamazepine or a carbamazepine-like compound to a subject in need thereof. Exemplary disorders include for example connective tissue and/or skeletal disorders.

### BACKGROUND TO THE INVENTION

There are a large number of phenotypically diverse disorders caused by inherited mutations of certain proteins. For example skeletal dysplasias are a group of over 450 clinically different and genetically heterogenous rare diseases. The translation of state-of-the-art technology into quantifiable patient benefits such as the development of new treatments has been extremely limited for genetic skeletal disorders (GSDs). The few notable exceptions include BMRN's drug candidate for ACH; a C-type natriuretic peptide (CNP) analogue PG-CNP37, bisphosphonate treatment for OI and fibrous dysplasia and enzyme replacement in lysosomal storage diseases or hematopoietic stem cell transplantation for infantile osteopetrosis.

More generally, many genetically inherited disorders have no treatment available and lifespan and quality of life may be considerably restricted.

It is an aim of the present invention to at least partly mitigate one or more of the above-mentioned problems.

It is an aim of certain embodiments of the present invention to provide a treatment for one or more inherited genetic disorders e.g. connective tissue and/or skeletal disorders.

It is an aim of certain embodiments of the present invention to provide a prophylactic treatment to prevent or reduce the likelihood of progression of a disorder such as a connective tissue or skeletal disorder in a patient identified as being at risk of developing such a disorder.

### DESCRIPTION OF CERTAIN EMBODIMENTS OF THE INVENTION

In a first aspect of the present invention, there is provided a composition comprising carbamazepine and/or a carbamazepine-like compound and/or a pharmaceutically acceptable salt or ester thereof for use in treating a disorder which is associated with and/or caused by an inappropriate intracellular accumulation of a protein, said inappropriate intracellular accumulation of the protein being caused by a mutation in the protein as compared to a wild-type version of the protein, wherein the disorder is selected from a skeletal disorder and a connective tissue disorder.

In a second aspect of the present invention, there is provided a method of treating a disorder which is associated with and/or caused by an inappropriate intracellular accumulation of a protein, said inappropriate intracellular accumulation of the protein being caused by a mutation in the protein as compared to a wild-type version of the protein, wherein the disorder is selected from a skeletal disorder and a connective tissue disorder, the method comprising providing a composition comprising carbamazepine and/or a carbamazepine-like compound and/or a pharmaceutically acceptable salt or ester thereof to a subject in need thereof. Aptly, the step of providing the composition comprises administering the composition to the subject.

Carbamazepine (CBZ) is an FDA approved drug for the use in treating epilepsy, bipolar disorder and neuropathic pain but also stimulates both autophagy and proteasomal degradation pathways and thereby prevented liver fibrosis occurring as a result of hepatocyte accumulation of misfolded mutant α1 antitrypsin. It is available in the U.S. as Tegretol® brand chewable tablets of 100 mg, tablets of 200 mg and suspension of 100 mg/5 mL, intended for oral administration. Carbamazepine for use in the methods and compositions of the present invention is commercially available. Alternatively, it may be synthesized by well-known methods, for example, that of U.S. Pat. No. 2,948,718.

Oxcarbazepine is an example of a carbamazepine-like compound which is also approved for use in treating epilepsy. Other CBZ-like compounds include CBZ metabolites, including but not limited to carbamazepine-10,11-epoxide and iminostilbene, as well as structurally related compounds, and oxcarbazepine metabolites, such as but not limited to 10,11-dihydro-10-hydroxy-carbamazepine (also known as “MHD”). Non-limiting examples of compounds structurally related to CBZ and OBZ include for example dihydro-CBZ, ethyl urea, phenyl urea, diphenylurea, dicyclohexylurea, phenytoin, substituted and unsubstituted iminobenzyl compounds, imipramine, (S)-(−)-10-acetoxy-10,11-dihydro-5H-dibenz[b,f]azepine-5-carboxamide (BIA 2-093), and 10,11-dihydro-10-hydroxyimino-5H-dibenz[b,f]azepine-5-carboxamide (BIA 2-024).

Thus, in certain embodiments, the carbamazepine-like compound is selected from oxcarbazepine, carbamazepine-10,11-epoxide and iminostilbene, as well as structurally related compounds, and oxcarbazepine metabolites, such as but not limited to 10,11-dihydro-10-hydroxy-carbamazepine (also known as “MHD”), dihydro-CBZ, ethyl urea, phenyl urea, diphenylurea, dicyclohexylurea, phenytoin, substituted and unsubstituted iminobenzyl compounds, imipramine, (S)-(−)-10-acetoxy-10,11-dihydro-5H-dibenz[b,f]azepine-5-carboxamide (BIA 2-093), and 10,11-dihydro-10-hydroxyimino-5H-dibenz[b,f]azepine-5-carboxamide (BIA 2-024).

Reference herein to carbamazepine and carbamazepine-like compounds encompasses compositions e.g. pharmaceutical compositions comprising such compounds.

Proteins are made inside a cell and they have to be folded into shape before they can carry out their function. They fold in the endoplasmic reticulum (ER). A mutation can alter the way a protein folds. If the protein cannot fold properly it remains inside the ER and cannot be secreted until it is correctly folded. In other words, the cells experiences “protein constipation” and the build up of unfolded proteins inside a cell causes stress to the ER. To help resolve ER stress, cells have evolved a mechanism called the unfolded protein response (UPR) which helps fold troublesome proteins into shape or be disposed of.

It is shown herein that CBZ is able to stimulate the intracellular degradation of mutant extracellular proteins e.g. collagen X proteins by either autophagy or proteasomal degradation depending on the particular mutation, leading to reductions in ER stress. In vivo, CBZ-induced reduction in ER stress allowed hypertrophic chondrocytes to improve their differentiation, resulting in increased bone growth rates and reduced skeletal dysplasia. The stimulation of intracellular proteolysis by CBZ may therefore be the first clinically viable way of treating the ER stress-associated disorders.

As used herein the terms “treatment” and “treating” refer to clinical intervention in an attempt to alter the natural course of the individual or cell being treated, and may be performed either for prophylaxis or during the course of clinical pathology. Desirable effects include preventing occurrence or recurrence of disease, alleviation of symptoms, diminishment of any direct or indirect pathological consequences of the disease, lowering the rate of disease progression, amelioration or palliation of the disease state, and remission or improved prognosis. For the avoidance of doubt, as used herein, the term “treatment” includes therapeutic and/or prophylactic treatment.

The composition may comprise one or more pharmaceutically acceptable excipients.

The composition may be formulated as a solid oral dosage form e.g. a tablet, a capsule or a caplet. In an alternative embodiment, the composition may be formulated for parental administration.

Aptly, the composition comprises carbamazepine and/or a carbamazepine-like compound in a therapeutically effective amount.

“Inappropriate intracellular accumulation” as used herein refers to an increased concentration of a protein e.g. an extracellular protein in a cell. Aptly, the inappropriate intracellular accumulation is caused by a mutation in a protein, e.g. an extracellular protein, as compared to a wild-type version of the protein.

In certain embodiments, the disorder is a disorder characterised by inappropriate accumulation of a mutant protein in a cell cytosol.

In certain embodiments, the disorder is a disorder characterised by inappropriate accumulation of a mutant protein in an endoplasmic reticulum of a cell. Aptly, the mutant protein is accumulated in a rough ER of a cell.

In certain embodiments, the disorder is characterised by accumulation of misfolded protein in the cell. Aptly, the misfolded protein is a mutant protein. In certain embodiments, the misfolded protein is accumulated in a cell cytosol. In certain embodiments, the misfolded protein is accumulated in an endoplasmic reticulum of a cell. Aptly, the misfolded protein is accumulated in a rough ER of a cell.

In certain embodiments, the disorder is characterised by an incomplete degradation of misfolded protein which is accumulated in a cell cytosol. That is to say in certain embodiments, CBZ can be used to treat disorders associated with partial but incomplete intracellular protein degradation by a cell, e.g. partial degradation of a mutant protein which is inappropriately accumulated in the cell.

In certain embodiments, the disorder is characterised by intracellular inclusion body formation/accumulation of a protein, e.g. a mutant protein. Aptly, the inclusion body formation/accumulation is in the cytosol e.g. the endoplasmic reticulum e.g. the rough endoplasmic reticulum of a cell.

In normal circumstances, i.e. the wild-type proteins, are not intracellular. The protein may be an extracellular protein or may be a cell membrane associated protein. Examples of membrane associated proteins include for example caveolin-3.

The protein may be a collagen, a glycoprotein or a proteoglycan for example.

In one embodiments, the protein is an extracellular matrix protein. The extracellular matrix (ECM) is a complex and dynamic network that surrounds cells in all tissues, providing structural and mechanical support, and mediating diverse biological processes that are crucial for supporting tissue formation and function.

“Extracellular matrix protein” as used herein refers to a protein which is contained in the extracellular matrix. Examples of extracellular matrix proteins include collagen proteins e.g. Type I collagen, Type II collagen, Type V collagen, Type VI collagen, Collagen X as well as for example COMP protein, aggrecan protein, fibulin-3, fibulin-5, cochlin, and uromodulin.

In certain embodiments, the composition is for use in treating a disorder which is caused by or associated with a mutation e.g. an inherited mutation, in one or more of the following proteins:
a) COMP (UniProtKB P49747 (COMP_HUMAN);
b) aggrecan (UniProtKB P16112 (PGCA_HUMAN);
c) matrillin-3 (UniProtKB 015232 (MATN3_HUMAN);
d) Type I collagen (UniProtKB P02452(CO1A1_HUMAN) and P08123 (CO1A2_HUMAN);
e) Type II collagen (UniProtKB P02458 (CO2A1_HUMAN);
f) fibulin-3 (UniProtKB Q12805 (FBLN3_HUMAN);
g) fibulin-5 (UniProtKB Q9UBX5 (FBLN5_HUMAN);
h) cochlin (UniProtKB O43405 (COCH_HUMAN);
i) Type IV collagen (UniProtKB-P02462 (CO4A1_HUMAN) and UniProtKB) P08572 (CO4A2_HUMAN) and UniProtKB Q01955 (CO4A3_HUMAN) and UniProtKB)-P53420 (CO4A4_HUMAN) and UniProtKB-P29400 (CO4A5_HUMAN) and UniProtKB)-Q14031 (CO4A6_HUMAN);
j) uromodulin (UniProtKB-P07911 (UROM_HUMAN)
k) caveolin-3 (UniProtKB-P56539 (CAV3_HUMAN));
l) rhodopsin UniProtKB (P08100 (OPSD_HUMAN));
m) Type XII collagen Q99715 (COCA1_HUMAN);
n) Type X collagen (UniProtKB-Q03692 (COAA1_HUMAN);
o) Type V collagen UniProtKB-P20908 (CO5A1_HUMAN) and UniProtKB-P05997 (CO5A2_HUMAN) and UniProtKB-P25940 (CO5A3_HUMAN); and
p) Type VI collagen UniProtKB-P12109 (CO6A1_HUMAN) and UniProtKB-P12110 (CO6A2_HUMAN) and UniProtKB-P12111 (CO6A3_HUMAN) and UniProtKB A2AX52 (CO6A4_MOUSE) and UniProtKB-A8TX70 (CO6A5_HUMAN) and UniProtKB-A6NMZ7 (CO6A6_HUMAN);

As used herein, the term “subject” refers to a human or animal subject.

In some embodiments, the subject exhibits characteristics such as for example short stature e.g. dwarfism. The subject may also exhibit characteristics such as facial dysmorphism.

As used herein, the term “skeletal disorder” is a disorder which affects the skeletal system of a subject. The skeletal system comprises bones and cartilage, tendon and ligament. The disorder treated by embodiments of the present invention may be caused by inappropriate intracellular protein accumulation in cells such as for example chondrocytes, osteoblasts, osteocytes, tendon and ligament cells. In certain embodiments, such cells have some protein degradation mechanism which causes a partial and incomplete degradation of the inappropriate intracellularly accumulated mutant protein.

In certain embodiments, the disorder may be a connective tissue disorder. As such, a disorder which affect cells making up cartilage which supports bone may be classified as both a skeletal disorder and a connective tissue disorder. A connective tissue disorder aptly affects one or more connective tissue components e.g. collagen, cartilage and other components which provide support in skin, tendons, ligaments, blood vessels, internal organs and bones. In certain embodiments, such cells has some protein degradation mechanism which causes a partial and incomplete degradation of the inappropriate intracellular accumulated mutant protein.

In certain embodiments, the disorder is an inherited skeletal and/or connective tissue disorder.

It will be appreciated that certain mutations in the proteins discussed herein may result in a subject presenting a plurality of phenotypes affecting a variety of organs.

In certain embodiments, the disorder is caused by or associated with an inherited mutation in a fibulin-5 protein as compared to a wild-type fibulin-5 protein. An example of an inherited disorder is autosomal dominant cutis laxa-2. Subjects suffering from autosomal dominant cutis laxa-2 may present with symptoms such as early childhood-onset pulmonary emphysema, peripheral pulmonary artery stenosis, and other evidence of a generalized connective disorder such as inguinal hernias and hollow viscus diverticula (e.g., intestine, bladder). Thus, in certain embodiments, carbamazepine and/or a carbamazepine-like compound and/or a pharmaceutically acceptable salt or ester thereof is for use in the treatment of autosomal dominant cutis laxa-2.

In certain embodiments, the connective tissue disorder is hereditary neuropathy with or without age-related macular degeneration (HNARMD). Optionally the disorder is caused by or associated with a mutation in a fibulin-5 protein. Thus, in certain embodiments, carbamazepine and/or a carbamazepine-like compound and/or a pharmaceutically acceptable salt or ester thereof is for the treatment of HNARMD.

In certain embodiments, the connective tissue disorder is a Type VI collagen disorder i.e. a disorder caused by or associated with a mutation in a Type VI collagen protein. Such Type VI collagen disorder include for example Bethlem myopathy which may characterized by the combination of proximal muscle weakness and variable contractures, affects most frequently the long finger flexors, elbows, and ankles. Onset may be prenatal (characterized by decreased fetal movements), neonatal (hypotonia or torticollis), in early childhood (delayed motor milestones, muscle weakness, and contractures), or in adulthood (proximal weakness and Achilles tendon or long finger flexor contractures). Other Type VI collagen disorders include for example Ullrich dystrophy which may be characterised by congenital weakness and hypotonia, proximal joint contractures and hyperlaxity of distal joints.

Thus, in certain embodiments, carbamazepine and/or a carbamazepine-like compound and/or a pharmaceutically acceptable salt or ester thereof is for use in the treatment of a Type VI collagen disorder e.g. a disorder selected from Bethlem myopathy and Ullrich dystrophy. In certain embodiments, the patient is a neonate or a child.

In certain embodiments, the connective tissue disorder is a Type V collagen disorder, i.e. a disorder which is caused by or associated with a mutation in a Type V collagen protein. Aptly, the disorder is inherited. Examples of such disorders include for example Ehlers-Danlos syndrome. Subjects typically suffer from joint hypermobility, hyperextensible and fragile skin which can split easily and wounds can be slow to heal and leave distinctive widened scars. Fragile and extensible tissues can also result in hernias, prolapse and cervical insufficiency.

In certain embodiments, the disorder is a subtype of Ehlers-Danlos syndrome (EDS). Non-limiting examples of Ehlers-Danlos subtypes include cardiac-valvular EDS, Vascular EDS, vascular-like EDS, kyphoscoliotic EDS, musculocontractual EDS, spondylocheirodysplastic EDS, brittle cornea syndrome, athrochalasis EDS, EDS/Osteogensis imperfecta overlap and dermatosparaxis EDS. Subjects typically suffer from similar symptoms as with EDS but subjects may also suffer from, for example, muscular hypotonia, cardiac valve insufficiency, arterial rupture, Caffey disease, bilateral hip dysplasia, kyphoscoliosis, muscle weakness and distal contractures.

Thus, in certain embodiments, the composition comprising carbamazepine and/or a carbamazepine-like compound and/or a pharmaceutically acceptable salt or ester thereof is for use in the treatment of a Type V collagen disorder e.g. EDS.

In certain embodiments, the protein is COMP. Aptly, the skeletal disorders are pseudoachondroplasia and multiple epiphyseal dysplasia and carbamazepine and/or a carbamazepine-like compound and/or a pharmaceutically acceptable salt or ester is for use in the treatment of pseudoachondroplasia and multiple epiphyseal dysplasia in a patient in need thereof. Aptly, the COMP protein comprises a mutation which causes deletion of aspartic acid at position 469. Other mutations may cause these disorders and are encompassed by certain embodiments of the present invention.

In certain embodiments, the skeletal disorder is multiple epiphyseal dysplasia and carbamazepine and/or a carbamazepine-like compound and/or a pharmaceutically acceptable salt or ester thereof is for use in the treatment of multiple epiphyseal dysplasia. Aptly, the multiple epiphyseal dysplasia may be dominant multiple epiphyseal dysplasia or may be recessive multiple epiphyseal dysplasia. Aptly, the COMP protein comprises a mutation which causes replacement of threonine at position 585 with methionine. Other mutations in the protein may cause these disorders and are encompassed by certain embodiments of the present invention.

In certain embodiments, the skeletal disorder is a disorder associated with or caused by a mutation in Collagen X. Aptly, the disorder is metaphyseal chondrodysplasia type Schmid (MCDS). In one embodiment, the disorder is not MCDS.

Mutations in collagen X cause the short limbed dwarfism metaphyseal chondrodysplasia type Schmid (MCDS). Mutant collagen X proteins misfold and are retained within the endoplasmic reticulum (ER) of hypertrophic chondrocytes, causing an increase in ER stress and activation of the unfolded protein response (UPR), ultimately disrupting the process of endochondral ossification. Increased ER stress in hypertrophic chondrocytes is the main cause of the MCDS pathology.

In certain embodiments, the protein is aggrecan. Aptly, the skeletal disorder is aggrecanopathy. In certain embodiments, the disorder is spondyloepimetaphyseal dysplasia. Spondyloepimetaphyseal dysplasia may be caused by a mutation in the gene (AGC1) which encodes aggrecan. Aptly, the mutation is a substitution of aspartic acid with asparagine at position 2381 of the aggrecan protein. Other mutations of the protein may also cause the disorder and are encompassed by certain embodiments of the invention. Aptly, the composition comprising carbamazepine and/or a carbamazepine-like compound and/or a pharmaceutically acceptable salt or ester thereof for is for use in the treatment of a skeletal disorder selected from aggrecanopathy and spondyloepimetaphyseal dysplasia.

Other types of spondyloepimetaphyseal dysplasia may be caused by a mutation in the gene which encodes Type II collagen.

In certain embodiments, the disorder is multiple epiphyseal dysplasia. Certain types of this disorder may be caused by a mutation in other genes such as for example matrillin-3.

In certain embodiments, the disorder is dominant osteochondritis dissecans. In certain embodiments, the composition comprising carbamazepine and/or a carbamazepine-like compound and/or a pharmaceutically acceptable salt or ester thereof is for use in the treatment of dominant osteochondritis dissecans.

A characteristic feature of dominant osteochondritis dissecans is areas of lesions caused by detachment of cartilage and a piece of the underlying bone from the end of the bone at a joint. People with this condition typically develop multiple lesions that affect several joints, primarily the knees, elbows, hips, and ankles. The lesions cause stiffness, pain, and swelling in the joint. Other characteristic features of familial osteochondritis dissecans include short stature and development of an osteoarthritis at an early age. Aptly, the disorder is caused by substitution of valine with methionine at position 2417 of the aggrecan protein. Other mutations in the protein may cause these disorders and are encompassed by certain embodiments of the present invention.

In certain embodiments, the composition is for use in treating osteogenesis imperfecta. Aptly, the disorder is caused by a mutation in Type I collagen. Aptly, the disorder is caused by one or more mutations in the Type I collagen protein. Subjects which suffer from Type I osteogenesis imperfecta may suffer from brittle bones, i.e. bones which fracture more easily than subjects which do not suffer from osteogenesis imperfecta.

In certain embodiments, the disorder is a Type II collagenopathy. The term “Type II collagenopathy” relates a plurality of disorders which have phenotypes such as for example disproportionately short stature, eye abnormalities, cleft palate, and hearing loss. Other phenotypes include for example degenerative joint disease such as arthritis, e.g. premature arthritis.

In certain embodiments, the disorder is a disorder caused by or associated with a mutation in Type XII collagen.

Aptly, the disorder is Ehlers-Danlos syndrome (EDS) and/or myopathy overlap syndrome. In certain embodiments, the disorder a subtype of EDS.

In certain embodiments, the disorder is caused by or associated with a mutation in uromodulin. Aptly, the disorder is an inherited disorder. The disorder may be for example a uromodulin-associated kidney disorder e.g. familial glomerulocystic kidney disease (GCKD) with hyperuricemia and isosthenuria, hyperuricemic nephropathy and/or medullary cystic kidney disease. Aptly, the disorder is caused by or associated with a mutation in a uromodulin protein which substitutes cysteine with tryptophan at position 147. Other mutations may cause these disorders and are encompassed by certain embodiments of the present invention.

In certain embodiments, the disorder is caused by or associated with a mutation in cochlin. Aptly, the disorder is an inherited disorder. In certain embodiments, the disorder is a disorder which affects the inner ear e.g. the cochlear. Aptly, the disorder is autosomal dominant sensorineural deafness (DFNA9).

In certain embodiments, the disorder is a disorder which affects the eye and/or supporting structures. Aptly, the disorder is causes retinal degeneration.

Aptly, the disorder is caused by a mutation in Type IV collagen. Thus, in certain embodiments carbamazepine and/or a carbamazepine-like compound and/or a pharmaceutically acceptable salt or ester thereof is for use in treatment of a Type IV collagen disorder. For example, the disorder is anterior segment dysgenesis and glomerulopathy and the composition comprising CBZ or the CBZ-like compound is for use in the treatment of anterior segment dysgenesis and glomerulopathy.

In certain embodiments, the disorder is age-related macular degeneration and carbamazepine and/or a carbamazepine-like compound and/or a pharmaceutically acceptable salt or ester thereof is for use in the treatment of age-related macular degeneration.

In certain embodiments, the disorder is autosomal dominant maculopathy. Aptly, autosomal dominant maculopathy is caused by or associated with a mutation in fibulin-3 protein. Aptly, the mutation is a single missense mutation (Arg345Trp; R345W). Other disorders which may be caused by or associated with the mutation of a fibrulin-3 protein as compared to wild-type include for example malattia Leventinese and Doyne honeycomb retinal degeneration. These disorders are typically characterized by the development of small round white spots (drusen) involving the posterior pole of the eye, including the areas of the macula and optic disc which appear in early adult life. Progression to form a mosaic pattern may follow. The disorders are characterised by slowly progressive loss of central visual acuity. However, the clinical course may change to one of very rapid progression and severe visual loss if choroidal neovascularization invades the subretinal space.

In certain embodiments, the disorder is caused by or associated with a mutation in a rhodopsin protein. Aptly, the disorder is a heritable retinitis pigmentosa. Aptly, the disorder is characterised by general retinal cell death. Aptly, the disorder is congenital stationary night blindness.

In certain embodiments, the disorder is caveolinopathy. Caveolinopathy may be considered both a skeletal and a cardiac disorder. Phenotypes that have so far been associated with primary caveolin-3 deficiency include limb girdle muscular dystrophy and cardiomyopathy. In certain embodiments, caveolinopathy may be associated with a mutation in a caveolin-3 protein which substitutes proline with leucine at position 104. Other mutations may cause these disorders and are encompassed by certain embodiments of the present invention.

In certain embodiments, the disorder is a disorder which affects the cardiac system of a subject. Aptly, the disorder is a disorder which affect small blood vessels. Aptly, the disorder is a disorder which affects one or more cardiac valves.

In certain embodiments, carbamazepine and/or a carbamazepine-like compound and/or a pharmaceutically acceptable salt or ester thereof is for use in the treatment of haemorrhagic stroke as a result of a mutation in Type IV collagen. In certain embodiments, the disorder is haemorrhagic stroke as a result of an inherited mutation in a Type IV collagen. In certain embodiments, these disorders affect the basement membrane of a subject. Mutations in the COL4A1 have been linked to disorders of the small blood vessels.

In certain embodiments, the mutation is a COL4A1 or a COL4A2 missense mutation and the disorder is caused by or associated with a COL4A1 or a COL4A2 missense mutation.

Without being bound by theory, it is considered that certain mutations in Type IV collagen may be linked to or increase the risk of haemorrhagic stroke in a subject. Therefore, subjects with one or more mutations in Type IV collagen may have an increased risk of haemorrhagic stroke. Some mutations of Type IV collagen may result in patients having phenotypes of differing severity.

In certain embodiments, the disorder is caused by a G702D mutation in COL4A2 in the subject.

In certain embodiments, carbamazepine and/or a carbamazepine-like compound and/or a pharmaceutically acceptable salt or ester thereof is for use in the treatment or prevention of cerebral haemorrhage without or without porencephaly in infants. In certain embodiments, the compound is for use in the treatment of haemorrhagic stroke in adults.

Haemorrhagic stroke accounts for between 10-15% of all strokes and yet account for a majority of deaths resulting from stroke. Furthermore, severe impairment can result from haemorrhagic stroke. Thus, prevention of such strokes may be particularly beneficial.

In a further aspect of the present invention there is provided a method of preventing and/or lessening the likelihood of haemorrhagic stroke in a subject at risk thereof, the method comprising providing a composition comprising carbamazepine and/or a carbamazepine-like compound and/or a pharmaceutically acceptable salt or ester thereof to a subject in need thereof.

Aptly, the step of providing the composition comprises administering the composition to the subject. The composition may be administered e.g. once a day.

In one aspect of the present invention there is provided a composition comprising carbamazepine and/or a carbamazepine-like compound and/or a pharmaceutically acceptable salt or ester thereof for use in preventing and/or lessening the likelihood of haemorrhagic stroke.

In certain embodiments, the subject has been identified as having a mutation in a Col4A1 and/or Col4A2 gene.

In certain embodiments, the method further comprises screening a subject for a Col4A1 gene mutation and/or a Col4A2 gene mutation.

In a further aspect of the present invention, there is provided carbamazepine and/or a carbamazepine-like compound and/or a pharmaceutically acceptable salt or ester thereof for use in prophylactic treatment of a Collagen IV disorder. As used herein, the term “prophylactic” treatment refers to prevention of a Collagen IV disorder or a reduction in the likelihood of a subject suffering from symptoms resulting from a Collagen IV disorder. In certain embodiments, the administration of carbamazepine and/or a carbamazepine-like compound to a subject may slow down or retard progression of a Collagen IV disorder.

In certain embodiments, the disorder is inherited autosomal dominant porencephaly type I. Aptly, the disorder is caused by a mutation in Type IV collagen e.g. a substitution of glycine with aspartic acid at position 702 in the protein. Other mutations may cause these disorders and are encompassed by certain embodiments of the present invention. Inherited autosomal dominant porencephaly type I is associated the development of fluid-filled cysts and cavities on the surface of the brain. Other disorders which are associated with Type IV collagen mutations which may be treated (including prophylactic treatment) by the composition described herein include for example brain small vessel disease with hemorrhage and HANAC (hereditary angiopathy with neuropathy, aneurysms, and muscle cramps) syndrome. In certain embodiments, the composition comprising carbamazepine and/or a carbamazepine-like compound and/or a pharmaceutically acceptable salt or ester thereof is for use in the treatment of brain small vessel disease with hemorrhage and HANAC (hereditary angiopathy with neuropathy, aneurysms, and muscle cramps) syndrome in a subject.

HANAC syndrome is a hereditary disorder which may be caused by one or more mutations in the COL4A1 gene and/or Type IV collagen protein. HANAC and its symptoms are often associated with weak basement membranes and/or blood vessels which are susceptible to damage. Subjects may suffer from nephropathies such as red blood cells in the urine (haematuria) and formation of cysts on one or more of the kidneys. Subjects may also suffer from changes in the white matter tissue of the brain, which may be referred to as leukoencephalopathy and aneurisms. HANAC may also cause muscle cramps which can occur in any muscle of a subject and may last from minutes to hours. Cramps may be spontaneous or exercise induced. Subjects may also suffer from eye related conditions such as, for example, arterial retinal tortuosity, episodes of bleeding in the eye, temporary loss of vision, cataracts, underdevelopment of the iris and an “off-centred” retina.

In some embodiments, the disorder is HANAC or another Col4A1 disorder. In certain embodiments, the HANAC disorder affects the kidney, muscle, and cardiovascular system, including retinal and cerebral vessels. Typically, HANAC syndrome affects the basement membranes.

In certain embodiments, the disorder is a disorder other than a HANAC syndrome which affects basement membranes. Basement membranes are formed from condensed networks of extracellular matrix (ECM) proteins. These structures underlie all epithelial, mesothelial and endothelial sheets and provide an essential structural scaffold. Disorders other than a HANAC syndrome which affect basement membranes may include but are not limited to Alport's syndrome, Dystrophic epidermolysis bullosa, junctional epidermolysis bullosa and Knobloch syndrome.

The practice of embodiments of the present invention will employ, unless otherwise indicated, conventional techniques of chemistry, molecular biology, pharmaceutical formulation, pharmacology and medicine, which are within the skill of those working in the art.

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Most general pharmaceutical formulation techniques can be found in Pharmaceutical Preformulation and Formulation (2^{nd} Edition edited by Mark Gibson) and Pharmaceutical Excipients: Properties, Functionality and Applications in Research and Industry (edited by Otilia M Y Koo, published by Wiley).

Most general pharmacological techniques can be found in A Textbook of Clinical Pharmacology and Therapeutics (5^{th} Edition published by Arnold Hodder).

Most general techniques on the prescribing, dispensing and administering of medicines can be found in the British National Formulary 72 (published jointly by BMJ Publishing Group Ltd and Royal Pharmaceutical Society).

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2^{nd} ed., 2002, CRC Press; The Dictionary of Cell and Molecular Biology, 3^{rd} ed., Academic Press; and the Oxford University Press, provide a person skilled in the art with a general dictionary of many of the terms used in this disclosure. For chemical terms, the skilled person may refer to the International Union of Pure and Applied Chemistry (IUPAC).

Units, prefixes and symbols are denoted in their Systeme International d'Unités (SI) accepted form. Numeric ranges are inclusive of the numbers defining the range.

The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. pH buffering agents may be phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, arginine, lysine, or acetate or mixtures thereof. The term further encompasses any agents listed in the US Pharmacopeia for use in animals, including humans.

The term “pharmaceutically acceptable salt” refers to a salt of a compound disclosed herein. Salts include pharmaceutically acceptable salts such as acid addition salts and basic salts. Examples of acid addition salts include hydrochloride salts, citrate salts and acetate salts. Examples of basis salts include salts where the cation is selected from alkali metals, such as sodium and potassium, alkaline earth metals, such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl. Other examples of pharmaceutically acceptable salts are described in “Remington's Pharmaceutical Sciences”, 17^{th} edition. Ed. Alfonoso R. Gennaro (Ed.), Mark Publishing Company, Easton, Pa., U.S.A., 1985 and more recent editions, and in the Encyclopaedia of Pharmaceutical Technology.

The term “solvate” in the context of the present disclosure refers to a complex of defined stoichiometry formed between a solute and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

“Treatment” is an approach for obtaining beneficial or desired clinical results. For the purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures in certain embodiments. Those in need of treatment include those already with the disorder as well as those in which the disorder is to be prevented. By treatment is meant inhibiting or reducing an increase in pathology or symptoms when compared to the absence of treatment, and is not necessarily meant to imply complete cessation of the relevant condition.

The pharmaceutical compositions comprising carbamazepine can be in unit dosage form. In such form, the composition is divided into unit doses containing appropriate quantities of the active component. the unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. In certain embodiments, packaged forms include a label or insert with instructions for use. Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, and transdermal) administration. The formulations may conveniently be presented in unit dosage form and may be prepared by any of the methods well known in the art of pharmacy.

As used herein an “effective” amount or a “therapeutically effective amount” of a compound or agent refers to a nontoxic but sufficient amount of the compound or agent to provide the desired effect. The amount that is “effective” will vary from subject to subject, depending on the age and general condition of the individual, mode of administration, and the like. An appropriate “effective” amount in any individual case may be determined by one of ordinary skill in the art using routine experimentation.

The terms “patient”, “subject” and “individual” may be used interchangeably and refer to either a humans or non-human mammal. Aptly, the subject is a human. As described herein, the subject may be a subject who has been identified as having one or mutations in a gene disclosed herein.

### DETAILED DESCRIPTION OF EMBODIMENTS OF THE INVENTION

Certain embodiments of the present invention are detailed below, by way of example only, with reference to the accompanying Figures in which:

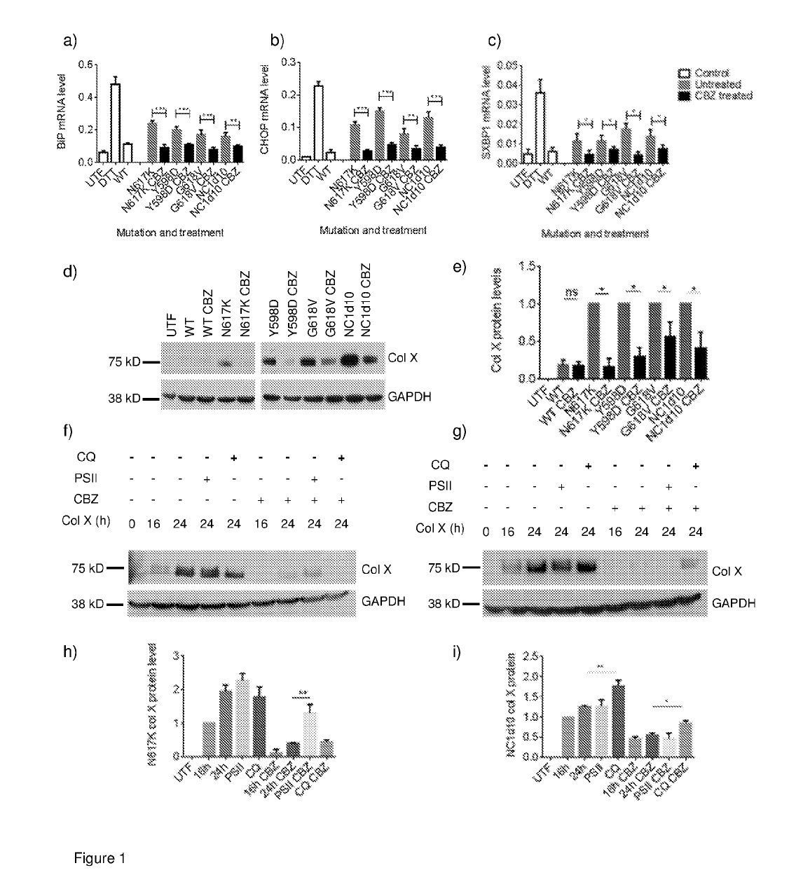

FIG. 1 illustrates the effects of carbamazepine treatment on ER stress induced by four different MCDS-causing mutations in collagen X; (a) BiP, b) CHOP c) spliced XBP1 mRNA level in cells transiently expressing one of four mutant collagen X constructs and treated for 24 hours with Carbamazepine (CBZ) (20 μM). Quantitation is relative to actin mRNA levels. Mean±SEM. (d) Western blot showing intracellular collagen X protein levels with and without 24 hours CBZ treatment. Quantification of three independent experiments shown in (e). (f) Western blot for N617K collagen X collagen X protein levels in the presence of CBZ for either 0 or 24 hours. Inhibitors of the proteasome (PSII) or lysosome (Chloroquine CQ) were added at 16 h post transfection for a further 8 hours. (g) Western blot of Nc1df10 collagen X using the same inhibitors as in (f). Quantification of three independent experiments relative to GAPDH and standardised against the protein level in the 16 h untreated timepoint is shown in (h) and (i). Mean±SEM of three independent experiments. (*P<0.05 **P<0.005 ***P<0.0005).

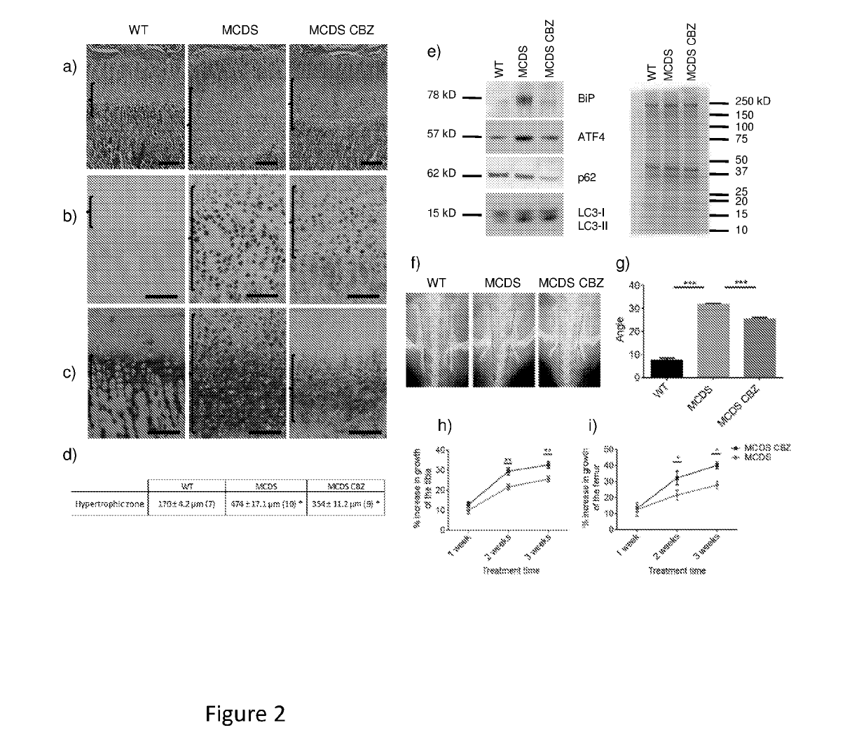

FIG. 2 illustrates the effect of carbamazepine on the growth plate pathology associated with MCDS. 3 week old MCDS mice were treated with CBZ for a period of one week. Untreated MCDS mice and mice wild-type for collagen X (WT) were used as controls. (a) H+E staining of the tibial growth plate at 4 weeks of age. (b) Immunohistochemistry for Grp78 and (c) Immunohistochemistry for collagen X. Yellow bars show the hypertrophic zone. Scale bars equal to 100 μM in all cases. (d) is a table showing growth plate zone measurements at 4 weeks of age. Mean±SEM. Number of animals shown in brackets. Significance between mutant untreated mice and treated mice shown by the asterisk. (e) Western blots of rib growth plate cartilage at 4 weeks of age for Grp78, ATF4, p62/SQSTM1 and LC3-II alongside the associated coomassie blue stain used as a loading control. Quantification of these blots against the coomassie protein level is shown in supplementary S4. (f) X-ray images of the pelvis at 6 weeks of age. The angle between the lines was measured in a minimum of 5 animals and a graph showing the mean±SEM is shown in (g). Graphs showing the percentage increase in growth of the (h) tibia and (i) femur from 3 weeks of age.

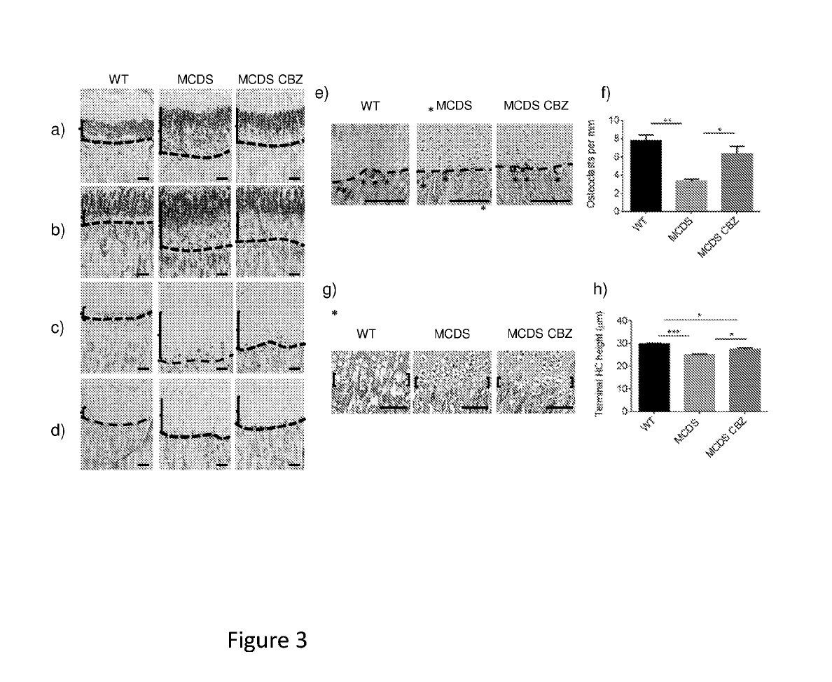

FIG. 3 illustrates the effect of carbamazepine on chondrocyte differentiation MCDS mice were treated with CBZ for 1 week. In situ hybridisation for a) collagen X mRNA levels b) Collagen II mRNA levels c) Osteopontin mRNA levels d) MMP13 mRNA levels. e) TRAP staining for osteoclast number, osteoclasts shown by the white asterisks. (f) Quantification of the number of osteoclasts per mm of vascular invasion front. Mean±SEM. (g) H+E image showing the terminal hypertrophic chondrocytes at the vascular invasion front. g) The height of the most terminal hypertrophic chondrocytes (within the red bracket) was measured, and mean±SEM is shown in (h). Hypertrophic zone marked by the yellow bar. Vascular invasion front shown by the orange line. Scale bar equal to 100 μM in all cases. Mean±SEM (*P<0.05 **P<0.005 ***P<0.0005).

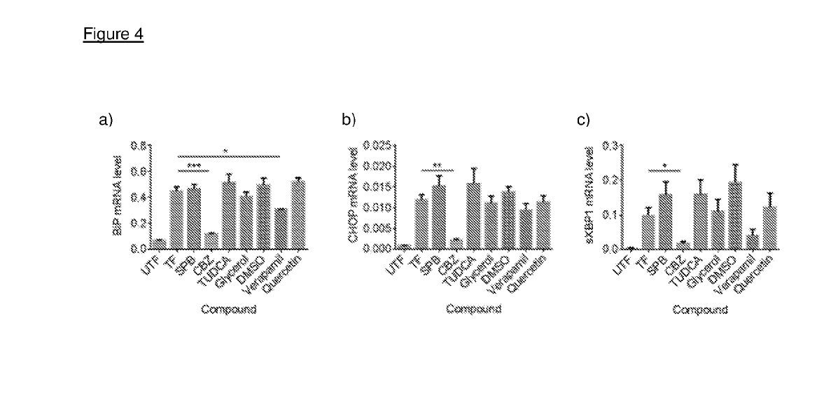

FIG. 4 Screen for compounds capable of reducing ER stress in vitro HeLa cells were transiently transfected with N617K collagen X and treated for 24 hours with the compounds indicated. UTF=untransfected control cells, TF=transfected control cells SPB=sodium phenyl butyrate CBZ=carbamazepine (20 μM), TUDCA=Tauroursodeoxycholic acid, DMSO=dimyethyl sulfoxide. RNA was extracted from cells and used for qPCR analysis. (a) BiP mRNA levels, (b) CHOP mRNA levels and (c) spliced XBP1 mRNA levels. Mean±SEM (n=3). (*P<0.05 **P<0.005***P<0.0005)

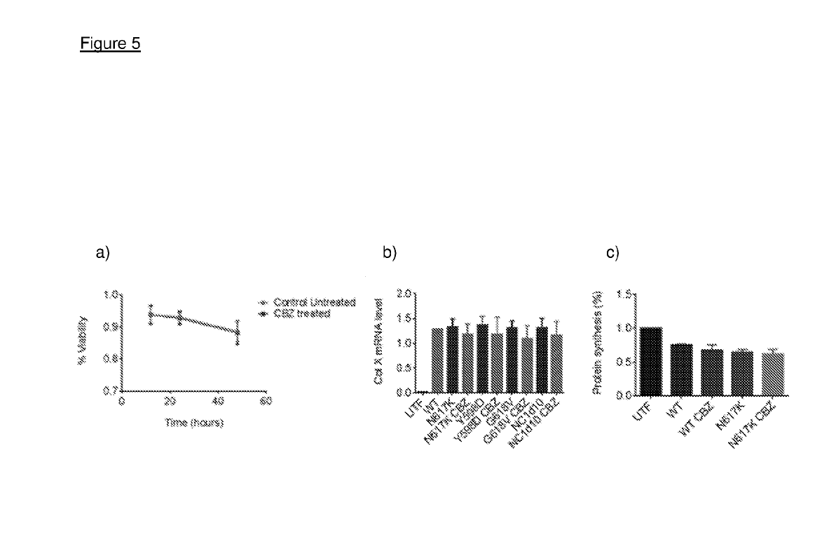

FIG. 5 illustrates the effect of CBZ treatment in vitro (a) Cell viability of cell treated with 20 μM CBZ. (b) Collagen X mRNA level of HeLa cells transiently transfected with a rang of collagen X mutations in the presence or absence of CBZ for 24 hours. (c) General protein synthesis rates of cells expressing wild-type (WT) or N617K collagen X and treated with CBZ for 24 hours. UTF=untransfected cell controls. Mean±SEM of three independent experiments in each case.

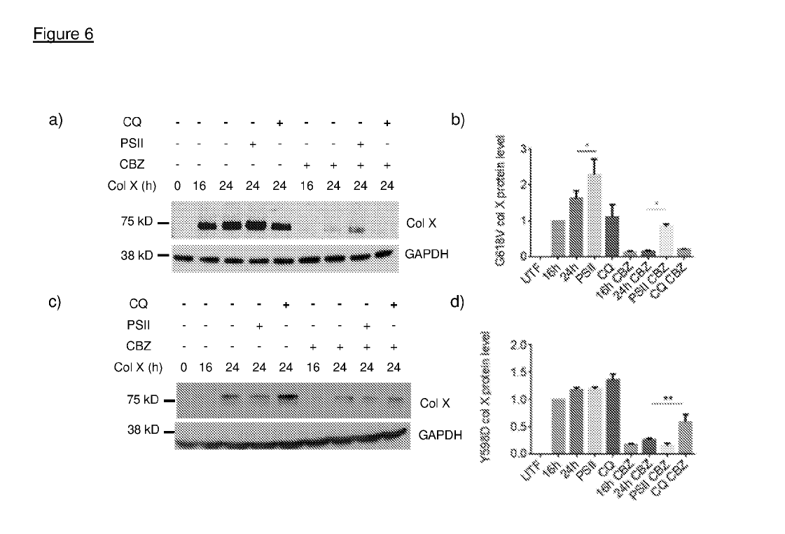

FIG. 6 illustrates the degradation route of mutant collagen X proteins Western blot for (a) G618V or (c) Y598D collagen X protein levels in the presence of CBZ for either 0 or 24 hours. Inhibitors of the proteasome (PSII) or lysosome (Chloroquine CQ) were added at 16 h post transfection for a further 8 hours. Quantification of three independent experiments relative to GAPDH and standardised against the protein level in the 16 h untreated timepoint is shown in (b) and (d). Mean±SEM of three independent experiments. (*P<0.05 **P<0.005).

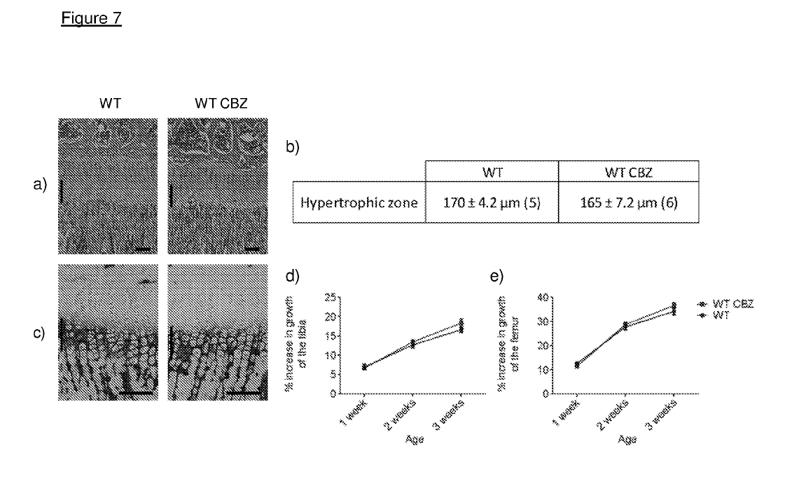

FIG. 7 illustrates the effect of CBZ treatment of WT mice Wild-type (WT) mice were treated with CBZ for a period of one week. Untreated mice were used as controls. (a)H+E images of the tibial growth plate at 4 weeks of age. (b) Table showing measurements of the growth plate zones. Mean±SEM. Number of animals shown in bracket. (c) Immunohistochemistry for collagen X in the tibial growth plate. Percentage increase in bone growth of the (d) Tibia and (e) Femur from 3 weeks of age. Mean±SEM.

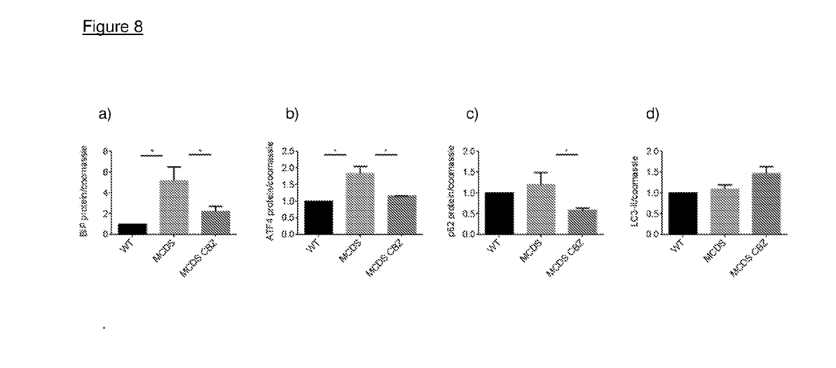

FIG. 8 illustrates the quantification of western blots on mouse rib growth plate tissue MCDS mice were treated with CBZ for a period of one week. Untreated MCDS mice and mice wild-type for collagen X (WT) were used as controls. Rib cages were dissected, treated with collagenase for 2 hours and cartilage growth plates were further dissected from each individual rib. Growth plates were pooled from 3 individual mice and protein extracted in SDS-PAGE buffer. 20 μg was used in SDS-PAGE and western blotting. Quantification of (a) BiP, (b) ATF4, (c) p62 and (d) LC3B protein levels against the coomassie protein level. Mean±SEM of three independent experiments;

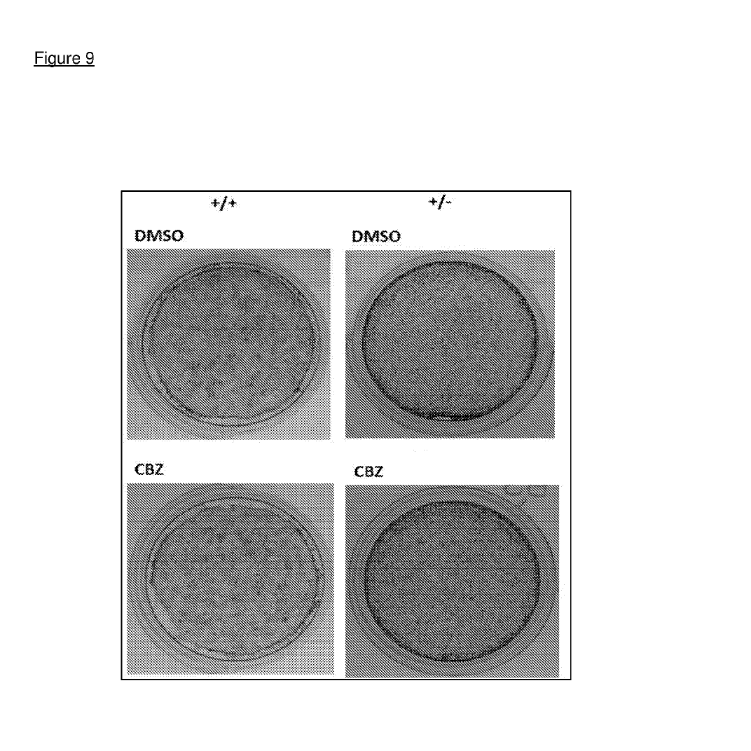

FIG. 9 illustrates comparison of qualitative changes in alkaline phosphatase activity in Col1a2 p.G610C mice osteoblast cells and wild type osteoblast cells. Osteoblasts were cultured for 7 days in osteogenic differentiation media prior to fixation and staining. Photographs of 12-well plate wells containing wild type (+/+) and Col1a2 p.G610C (+/−) mice osteoblast cells untreated (DMSO) or treated with CBZ; and

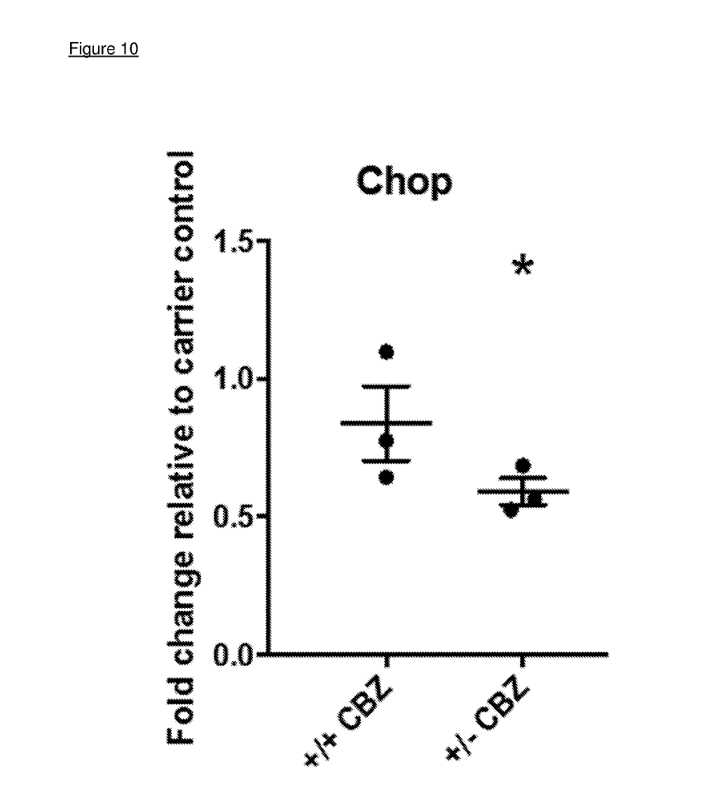

FIG. 10 illustrates the change in CHOP for wild (+/+) and Col1a2 p.G610C (+/−) mice osteoblast cells treated with CBZ. The graph shows the fold change in CHOP expression relative to carrier treated (DMSO) controls. Cells were incubated for 21 days with 20 μM CBZ before extracting RNA and quantification by qPCR. Each data point represents a separate biological replicate. Error bars show standard error of mean. *=p<0.05.

### EXAMPLES

Ethics Statement:

All mice used in this study were maintained, handled and sacrificed in strict accordance with UK Home Office regulations (PPL40/3485).

Antibodies

Antibodies used in this study were as follows: BiP (Santa-cruz sc-1051), ATF4 (Cell signalling 11815), LC3B (Sigma L7543), P62 (Santa-Cruz sc-28359), Anti-his (R+D systems MAB050). Collagen X (made in Ray-Boot Handfords lab, University of Manchester).

Constructs Used

pCEP4.WT.ColX, pCEP4.N617K.ColX, pCEP4.Y598D.ColX, pCEP4.G618V.ColX and pCEP4.NC1de110.ColX constructs were provided by John Bateman (Melbourne, Australia) (Wilson et al 2005).

### Example 1

In vivo Experiments

The generation and genotyping of the Col10a1 p.N617K mouse line has been described previously (Rajpar et al 2009). Mice homozygous for the MCDS-causing N617K collagen X mutation were x-rayed at 3 weeks of age and treated with carbamazepine (250 mg/kg/day) for up to 21 days via a subcutaneous implantation of a slow-release pellet (Innovative research of America USA C-113) whilst the mouse was under anaesthetic. The incision was sutured closed, glued using Vetbond tissue adhesive (Santa-Cruz sc-361931) and the mice given an appropriate dose of buprenorphine painkiller depending on their body weight. Male mice were X-rayed once per week in order monitor bone growth; female mice were sacrificed after 1 week of treatment either by cervical dislocation or by carbon dioxide overdose under the provisions of the Animals (Scientific Procedures) Act 1986 and tissues collected for histological analysis. All procedures were carried out according to Home Office regulations.

Skeletal Measurements

Mice were anaesthetised using isofluorane, placed on X-Ray hyperfilm (GE healthcare, GZ28906850) and X-Rayed using a Flaxitron X-ray specimen radiography system (Flaxitron MX-20). X-Rays were performed prior to implantation and once per week for a maximum of 3 weeks. The analysis of the skeleton was done using Growbase software (Certus Technology Associated Limited, UK) to calculate the length of the femur, tibia and inner canthal distance. The bone growth rate was determined as a percentage increase relative to measurements at 3 weeks of age. Image J was used to analyse the angle of deflection from the vertical tuberosity of the ischium to determine the severity of the hip dysplasia phenotype. All measurements were analysed by ANOVA to determine significance using GraphPad Prism 6.0 software.

Histology

Hind limbs were dissected and fixed overnight in ice-cold 4% paraformaldehyde (w/v) in 1×DEPC-PBS or 95% ethanol/5% acetic acid (v/v), and decalcified in 0.8 M ethyl-diamine tetracetic acid (EDTA) pH 7.4 for a period of one week. The samples were embedded in paraffin wax and sectioned sagittally using a cool-cut HM 355 S microtime (MicRom) generating 5 μm thick sections. Sections were collected on positively charged superfrost slides (VWR) and dried overnight prior to histological staining, immunohistochemistry or in situ hybridisation.

H+E Staining

H+E staining was performed as described previously ¹⁰ and images taken using a Carl Zeiss Axiovision microscope fitted with an Axiocam colour CCD camera and associated Axiovision software.

Growth plate zone widths were measured on images of known magnification as described previously (Rajpar et al 2009). The beginning of the proliferative zone was defined as the point at which the individual round chondrocytes flattened out and arranged into columns. The start of the hypertrophic zone was defined as the point at which proliferative chondrocytes rounded up and enlarged. The end of the hypertrophic zone was defined as the vascular invasion front. For each animal the data from three separate sections, spaced a minimum of 50 μm apart were averaged. Measurements were analysed for statistical significance by one-way ANOVA using GraphPad Prism 6.0 software.

Immunohistochemistry

Immunohistochemistry for collagen X and BiP was performed on 95% ethanol/5% acetic acid fixed tibial growth plate sections as described previously¹⁰. Images were captured using a Carl Zeiss Axiovision microscope fitted with an Axiocam colour CCD camera and associated Axiovision software.

In Situ Hybridisation (ISH)

DIG-labelled colourimetric ISH was performed as described previously⁶. The resulting cDNA probes were cloned into a pT7T3 vector then linearized and transcribed using the appropriate restriction enzyme and RNA polymerase.

Tartrate-Resistant Acid Phosphatase (TRAP) Staining

Osteoclasts were stained using a TRAP staining kit (Sigma-Aldrich 387A) according to manufacturer's instructions. The number of stained osteoclasts per mm of vascular invasion front was quantified in 3 sections per animal spaced 50 μm apart in 3 animals per genotype and analysed by one-way ANOVA for statistical significance.

Tissue Preparation for Use in Western Blotting

Rib-cages from 4 week old mice were dissected and placed in collagenase medium for 1-2 hours, after which the cartilage growth plates were collected using a dissecting microscope. Rib growth plates were then homogenized using a mikro-dismembranator in 100 ul of 2× sample buffer and protein extracted by boiling samples at 99° C. for 10 minutes and centrifuging at 13,000 rpm for 15 minutes at 4° C. The supernatant was collected and analysed by SDS-PAGE.

Cell Culture

HeLa cells were cultured at 37° C. with 5% CO₂ in Dulbecco's modified Eagle's medium (Life Technologies) containing 10% fetal bovine serum, penicillin (0.5 U/ml)/streptomycin (0.5 μg/ml), 2 mM L-glutamine, and 1% non-essential amino acids.

Cells were seeded at an appropriate density so that they were approximately 70% confluent on the day of transfection. The medium surrounding the cells was replaced with fresh culture medium supplemented with 50 μg/ml ascorbic acid and the cells transiently transfected with the collagen X constructs using Lipofectamine 2000 for a period of 24 hours. Transfected cells were treated with 20 uM CBZ 30 minutes after transfection for a period of 24 hours. Cells were also treated with 1 mM DTT for 24 hours as a positive control for ER stress.

Protein Extraction In Vitro

Protein was extracted from cells 24 hours post-transfection. Briefly media was removed and cells washed with 1×PBS. 100 μl of 2×SDS PAGE buffer was added per well of a 6 well plate and cells scraped into Eppendorf tubes. Samples were heated to 99° C. for 10 minutes and centrifuged at 13,000 rpm for 15 minutes at 4° C. The resulting supernatant was transferred to a fresh tube and protein concentration determined using a Pierce bicinchoninic acid (BCA) protein assay.

SDS-PAGE and Western Blotting

The protein concentration of samples was determined using a Pierce bicinchoninic acid (BCA) protein assay with a bovine serum albumin standard curve. 20 μg per sample was loaded into SDS-PAGE gels. Proteins were separated by SDS-polyacrylamide gel electrophoresis and transferred onto Hybond-P PVDF membrane (LC3) or nitrocellulose membrane (all other proteins). Membranes were blocked using 2% milk powder in PBS containing 0.1% tween-20 for 1 hour at room temperature. The membranes were incubated in primary antibody in blocking solution overnight at 4 degrees at a dilution according to manufacturer's guidelines. The membranes were washed three times in PBS-T and incubated with an appropriate HRP conjugated secondary antibodies in blocking buffer for 1 hour at room-temperature. Membranes were washed three times in PBST and developed using an ECL detection kit (Life Technologies) and ECL hyperfilm (GE healthcare).

The blots were quantified by densitometry analysis using ImageJ software, normalised to a loading control and standardised against a control sample on each blot. The results were analysed by ANOVA for statistical significance using Graph Pad Prism 6.0 software.

RNA Extraction

Media was discarded from wells and the cell layer washed with 1×PBS. 1 ml of TRIzol reagent (Life Technologies) was added per well of a 6 well plate and incubated for 5 minutes at room temperature. Cells were scraped into a 1.5 ml Eppendorf tube, 0.2 ml chloroform added and the tube mixed well. Samples were incubated for 3 minutes at room temperature and then centrifuged at 4° C. at 10,000 rpm for 20 minutes. The upper phase was transferred to a fresh Eppendorf and 0.5 ml isopropanol was added in order to precipitate the RNA. Samples were mixed well and incubated for 10 minutes at room temperature before being centrifuged at 10,000 rpm for 15 minutes at 4° C. The supernatant was carefully removed, taking care not to disturb the pellet. The pellet was washed in 70% ethanol and resuspended in 30 μl DEPC-H₂O.

RT-qPCR Analysis

RNA samples were DNase treated using the DNA free kit (Applied Biosystems, AM1906) according to manufacturer's instructions in order to remove genomic DNA. 1 μg of RNA was used for reverse transcription using the Taqman Reverse Transcription Reagent Kit (Life technologies UK, 8080234) according to manufactures instructions.

Real time qPCR was performed using the following primers: Collagen X forward 5′CTTCTTTCTCCTTTGCCTG3′ (SEQ. ID. NO: 1) and reverse 5′GCTCTCCTCCTTACTGCTATAC3′ (SEQ. ID. NO: 2), BiP forward 5′ GCTAATGCTTATGGCCTGGA3′ (SEQ. ID. NO: 3) and reverse 5′CGCTGGTCAAAGTCTTCTCC3′ (SEQ. ID. NO: 4), CHOP forward 5′ GCGCATGAAGGAGAAAGAAC3′ (SEQ. ID. NO: 5) and reverse 5′TCTGGGAAAGGTGGGTAGTG3′ (SEQ. ID. NO: 6), spliced XBP1 forward 5′ GAAGCCAAGGGGAATGAAGT3′ (SEQ. ID. NO: 7) and reverse 5′ CCAGAATGCCCAACAGGATA3′ (SEQ. ID. NO: 8) and β-actin forward 5′ CCACCATGTACCCAGGCATT3′ (SEQ. ID. NO: 9) and reverse 5′CACATCTGCTGGAAGGTGGA3′ (SEQ. ID. NO: 10). All reactions were performed in duplicate using a SYBR green kit on an ABIPrism™ 7000 sequence detector system (Applied Biosystems). Each experiment was performed three times in order to confirm the results. Significance determined by ANOVA using GraphPad Prism 6.0 software.

To determine the mRNA level of the spliced form of XBP1 the cDNA sample was subject to an initial 4 PCR cycles of 95° C. for 3 minutes, 4 cycles of 95° C. for 40 seconds, 60° C. for 45 seconds and 72° C. for 40 seconds, followed by 72° C. for 10 minutes in order to create double stranded cDNA. The double stranded cDNA was digested overnight at 37° C. using a Pst1 restriction enzyme in order to remove the unspliced form of XBP1. The resulting digested cDNA sample was then used in RT-qPCR as described above.

Results

The presence of increased ER stress in hypertrophic chondrocytes causes the cells to disengage their differentiation programme in an attempt to reduce ER stress and survive. The altered differentiation program results in a decreased level of VEGF being produced by hypertrophic chondrocytes, delaying vascular invasion and causing a characteristic expansion in the hypertrophic zone found in MCDS^{5,6}.

Several compounds were screened for their ability to reduce ER stress in cells expressing the MCDS-causing N617K collagen X mutation. CBZ was the only compound tested that reduced the mRNA levels for each of the ER stress markers BiP, CHOP and spliced XBP1 (FIG. 4). Furthermore, CBZ was able to significantly reduce the mRNA level of these stress markers in cells expressing 4 different MCDS-causing collagen X mutations (N617K, Y598D, G618V and NCdel10; FIG. 1a-c). For each of these mutations, the CBZ-induced reduction in ER stress was accompanied by significant reductions in the intracellular levels of mutant collagen X protein (FIG. 1d & e).

Next we focused on the rates of degradation to explain the CBZ-induced reduction in mutant protein accumulation since the drug did not affect cell viability, collagen X mRNA levels or rates of general protein synthesis (FIG. 5). The effects of both proteasomal (PSII) and autophagy (chloroquine) inhibitors on the accumulation of mutant collagen X in the cell culture system in the presence and absence of CBZ were tested. In the absence of CBZ, mutant collagen X accumulated over a 24 hour period (N617K & NCdel10, FIG. 1f-l; G618V & Y598D FIG. 5). For N617K and G618V, intracellular accumulation was enhanced by the proteasomal—but not by the autophagy—inhibitor which were added for the final 8 hours of culture. In contrast, the intracellular accumulation of the NCdel10 and Y598D was enhanced by the autophagy—but not the proteasomal—inhibitor. CBZ treatment did not alter the degradation pathway but rather enhanced the degradation rates for each of the 4 mutant collagen X proteins (FIG. 1f-l and FIG. 6). CBZ was therefore able to stimulate the degradation of mutant collagen X by both autophagy and the ER associated degradation/proteasome pathways.

In order to examine the effects of CBZ in vivo, 3 week old MCDS mice were treated with CBZ (250 mg/kg body weight/day) for 1 to 3 weeks. Untreated MCDS mice displayed the characteristic expansion in the size of the hypertrophic zone compared to WT mice. 1 week of CBZ treatment resulted in a significant reduction in the size of the hypertrophic zone compared to untreated MCDS controls (FIG. 2a, d). These data suggested that CBZ treatment was capable of reducing MCDS pathology since the degree of hypertrophic zone expansion positively correlates with disease severity CBZ treatment of WT mice had no effect on the hypertrophic zone width (FIGS. 8a and b) indicating that the drug was exerting its effects by alleviating the ER stress induced in the hypertrophic chondrocytes expressing mutant collagen X rather than by influencing normal growth plate differentiation.

Immunohistochemical analysis of tibial growth plates revealed, as expected, that WT collagen X was secreted into the extracellular matrix surrounding the hypertrophic chondrocytes whereas in MCDS mice, the mutant protein was retained intracellularly with a delayed and reduced collagen X secretion in the lower half of the hypertrophic zone (FIG. 2b). CBZ treatment reduced the level of intracellular collagen X in MCDS mice but there was no overt increase in the level of extracellular collagen X (FIG. 2b), suggesting that CBZ was stimulating the degradation of the intracellular mutant protein as observed in vitro. CBZ had no effect on collagen X protein localisation in WT mice (FIG. 8c).

MCDS mice displayed an increase in immunostaining for BiP protein within the hypertrophic zone of the tibial growth plate compared to WT mice (FIG. 2c) and a significant increase in the protein level of BiP and ATF4 in growth plate extracts analysed by western blotting (FIGS. 2e and 8), clearly indicating elevated ER stress. CBZ treatment reduced the BiP protein in growth plate sections of MCDS mice (FIG. 2b) and significantly reduced the levels of both BiP and ATF4 on western blots of growth plate extracts prepared from treated compared to untreated MCDS mice (FIGS. 2e and 8). In order to demonstrate that CBZ could exert a direct effect on growth plate cartilage we looked for two markers of autophagy in the growth plate extracts. CBZ treatment caused an increase in LC3B-II protein (converted from LC3B-I with activation of autophagy) and a decrease in the concentration of the autophagy substrate p62 (FIG. 2e) in growth plate extracts, indicating a stimulation of autophagy in the cartilage of treated mice. Therefore, CBZ treatment reduced ER stress in the cartilage of MCDS mice most likely by directly stimulating the proteolysis of mutant collagen X accumulating in the hypertrophic chondrocytes of MCDS mice.

One characteristic of the MCDS pathology in mouse is the presence of hip dysplasia-characterised by an increase in the angle of deflection of the ischial tuberosity⁶. 6 week old WT mice had an average deflection of 7.7° compared to 31.6° in the untreated MCDS mice (FIG. 2f, g). CBZ treatment for 3 weeks reduced this deflection to 25.4°, illustrating that relatively short periods of CBZ treatment have the capacity to improve the MCDS associated skeletal dysplasia (FIG. 2f, g). In addition, 3 weeks of CBZ treatment resulted in significant increases in the rates of long bone growth (FIGS. 2h & i). CBZ treatment in MCDS mice promoted 1.25 and 1.44 fold increases in the rates of tibial and femur bone growth in MCDS mice respectively after 3 weeks of treatment (mean±SEM, n=6, [% increase in bone length over treatment period] tibia: untreated 25.6±1.3 vs CBZ 32.7 ±1.6 p<0.05; femur: untreated 27.8±2.3 vs CBZ 40.1±2.0, p<0.05). CBZ treatment did not cause a significant increase in bone growth rates in WT mice (FIGS. 8d & e).

The altered differentiation of hypertrophic chondrocytes caused by increased ER stress in MCDS is characterised by a disrupted pattern of collagen X expression, a re-expression of collagen II and the sporadic expression of the terminal differentiation markers OPN and MMP13 by individual chondrocytes in the lower third of the hypertrophic zone as the synchronous process evident in the WT is lost (FIG. 3a-d). CBZ treatment altered the expression pattern of each of these markers towards that seen in the WT mouse (FIG. 3a-d). In addition, CBZ treatment partially restored the reduced osteoclast recruitment to the vascular invasion front (FIGS. 3e & f) and enhanced the hypertrophy, based on the height of the terminal hypertrophic chondrocytes, achieved in MCDS mice (FIG. 3g, h). These data clearly indicate that CBZ treatment shifts the disrupted hypertrophic differentiation apparent in MCDS toward the normal pattern seen in the WT mice.

In summary, CBZ treatment stimulates the degradation of intracellularly retained MCDS-causing mutant forms of collagen X by either autophagy or proteasomal pathways, reducing the level of ER stress both in vitro and in viva The CBZ-induced reduction in ER stress in turn causes less disruption to the differentiation process in hypertrophic chondrocytes leading to decreased skeletal dysplasia and increased bone growth rates. These data provide evidence for the CBZ-induced stimulation of intracellular proteolysis being a potential treatment strategy for reducing the clinical severity of MCDS and other disorders.

### Example 2

The effects of CBZ were studied on Calvarial osteoblasts from Col1a2 p.G610C mice lines (Osteogenesis Imperfecta mouse model).

Alkaline Phosphatase Activity Assay

For testing of Alkaline phosphatase activity wild type (+/+) and heterozygous ((+/−) from Col1 a2 p.G610C mice) osteoblasts were cultured for 7 days in osteogenic differentiation media and with DMSO (control) or CBZ prior to fixation and staining with an Alkaline phosphate staining kit as per the manufactures instructions.

Cellular Stress Assay

Expression levels of CCAAT-enhancer binding protein homologous protein (CHOP) was measured. Wild type (+/+) or Col1a2 p.G610C (+/−) cells were incubated in osteogenic differentiation media for 21 days with or without 20 μM CBZ.

RNA was extracted from the cells after 21 days and the levels of Chop gene expression was quantified by qPCR using a protocol as previously described herein.

Bone Quantity and Bone Composition Assay

Col1a2 p.G610C osteogenesis imperfecta mice lines are grown and treated with subcutaneous CBZ slow release pellet as described herein for Col10a1 p.N617K mouse lines or are grown for 5-6 weeks before treatment with intraperitoneal injections of increasing doses of CBZ. At 6 weeks of age subcutaneous implantation of a slow release pellet of CBZ is inserted into the skin.

Mice are then culled at 4-8 weeks and tissue from the femur, tibiae and vertebrae are collected from CBZ treated and untreated mice. Tissue samples are then compared using micro computed tomography.

Results

No change in ALP activity can be seen when either wild type or OI model cells are treated with CBZ (FIG. 9B), suggesting that CBZ treatment of osteoblasts does not interfere with osteoblast differentiation.

CBZ significantly decreased Chop expression (FIG. 10). As Chop expression is associated with cellular and ER stress and it is considered that a reduced level of CHOP suggests a reduced level of cell death. The results therefore indicate that CBZ may help reduce cell-stress related apoptosis.

### Example 3

The effect of CBZ on p.R992C Col2a1 mutant mice (spondyloepiphyseal dysplasia congenital (SEDC) model mice) was tested.

R992C Col2a1 SEDC mice were treated with carbamazepine (CBZ), to determine whether treatment ameliorates the disease phenotype. Increased chondrocyte apoptosis within the epiphyseal growth plate is associated with the onset of disease from 3 weeks in mouse models expressing this mutant form of COL2A1.

Chondrocyte apoptosis was detected by a Terminal deoxynucleotide transferase dUTP nick end labelling (TUNEL) assay. Tibial growth plates from wild type and SEDC mutant mice were stained according to the manufactures instructions before being imaged (data not shown).

Fluorescence microscopy images following TUNEL staining of the tibial growth plate from wild type and SEDC mutant mice treated with CBZ indicated that CBZ did not necessarily reduce the elevated level of apoptosis in tibial growth plate chondrocytes observed in SEDC mice suggesting that CBZ may be effective when treating a disorder characterised by cells which have at least some intrinsic protein degradation mechanism which may result in incomplete protein degradation.

However, quantitative analysis is not currently possible, since tissue from only a single treated wild type/SEDC mutant mouse is currently available.

### Example 4

Cells carrying a COL4A2 missense mutation (which causes haemorrhagic stroke) are treated with 50 μM CBZ. After treatment expression of BIP and ATF 6 are determined by qPCR and mRNA analysis and levels of BIP and ATF6 protein are determined by SDS-PAGE and western blot analysis as previously described herein.

It is shown that cells treated with CBZ have a decreased level of BIP and ATF6 expression and protein levels (data not shown). As both ATF 6 and BIP are markers of ER stress and increased expression is indicative of cell death experimental evidence indicates that CBZ may help decrease levels of mutated Type IV collagen in the ER and therefore CBZ may help to reduce ER stress and cell death. Therefore CBZ may also help to decrease the risk of and/or prevent haemorrhagic stroke.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.
1. Wallis, G. A., et al. Amino acid substitutions of conserved residues in the carboxyl-terminal domain of the alpha 1(X) chain of type X collagen occur in two unrelated families with metaphyseal chondrodysplasia type Schmid. Am J Hum Genet 54, 169-178 (1994).
2. Wallis, G. A., et al. Mutations within the gene encoding the alpha 1 (X) chain of type X collagen (COL10A1) cause metaphyseal chondrodysplasia type Schmid but not several other forms of metaphyseal chondrodysplasia. J Med Genet 33, 450-457 (1996).
3. Bateman, J. F., Wilson, R., Freddi, S., Lamande, S. R. & Savarirayan, R. Mutations of COL10A1 in Schmid metaphyseal chondrodysplasia. Hum Mutat 25, 525-534 (2005).
4. Cameron, T. L., et al. Transcriptional profiling of chondrodysplasia growth plate cartilage reveals adaptive ER-stress networks that allow survival but disrupt hypertrophy. PLoS One 6, e24600 (2011).
5. Tsang, K. Y., et al. Surviving endoplasmic reticulum stress is coupled to altered chondrocyte differentiation and function. PLoS Biol 5, e44 (2007).
6. Rajpar, M. H., et al. Targeted Induction of Endoplasmic Reticulum Stress Induces Cartilage Pathology. PLoS Genet 5, e1000691 (2009).
7. Sarkar, S., et al. Lithium induces autophagy by inhibiting inositol monophosphatase. J Cell Biol 170, 1101-1111 (2005).
8. Meng, Q., et al. Carbamazepine promotes Her-2 protein degradation in breast cancer cells by modulating HDAC6 activity and acetylation of Hsp90. Mol Cell Biochem 348, 165-171 (2011).
9. Hidvegi, T., et al. An autophagy-enhancing drug promotes degradation of mutant alpha1-antitrypsin Z and reduces hepatic fibrosis. Science 329, 229-232 (2010).
10. Kung, L. H., Rajpar, M. H., Preziosi, R., Briggs, M. D. & Boot-Handford, R. P. Increased Classical Endoplasmic Reticulum Stress Is Sufficient to Reduce Chondrocyte Proliferation Rate in the Growth Plate and Decrease Bone Growth. PLoS One 10, e0117016 (2015).

1-26. (canceled)

27. A method of treating a disorder associated with an inappropriate intracellular accumulation of protein, comprising:
administering a composition comprising carbamazepine and/or a carbamazepine-like compound and/or a pharmaceutically acceptable salt or ester thereof to a subject in need thereof;
wherein the inappropriate intracellular accumulation of the protein is caused by a mutation in the protein.

28. The method according to claim 27, wherein the carbamazepine-like compound is oxcarbazepine.

29. The method of claim 27, wherein the composition is administered orally or parentally.

30. The method of claim 27, wherein the disorder is associated with endoplasmic reticulum (ER) stress caused by the inappropriate intracellular accumulation of the protein.

31. The method of claim 27, wherein the extracellular protein is an extracellular matrix protein.

32. The method of claim 27, wherein the disorder is an inherited disorder.

33. The method of claim 27, wherein the disorder is pseudoachondroplasmia.

34. The method of claim 27, wherein the disorder is multiple epiphyseal dysplasia.

35. The method of claim 27, wherein the disorder is dominant osteochondritis dissecans.

36. The method of claim 27, wherein the disorder is spondyloepimetaphyseal dysplasia.

37. The method of claim 27, wherein the disorder is Type II collagenopathy.

38. The method of claim 27, wherein the disorder is osteogenesis imperfecta.

39. The method of claim 27, wherein the disorder is a Type IV collagen disorder selected from haemorrhagic stroke, inherited autosomal dominant porencephaly type I, HANAC syndrome and combinations thereof.

40. The method of claim 27, wherein the disorder is a Type IV collagen disorder selected from Bethlem and Ullrich dystrophies.

41. The method of claim 27, wherein the disorder affects the eye and/or its supporting structures.

42. The method of claim 41, wherein the disorder is anterior segment dysgenesis and glomerulopathy.

43. The method of claim 41, wherein the disorder is age-related macular degeneration.

44. The method of claim 27, wherein the disorder is heritable retinitis pigmentosa.

45. The method of claim 27, wherein the subject is under the age of 10 years.

46. The method of claim 27, wherein the subject is a pregnant female.
